(19) United States
(12) Patent Application Publication
(10) Pub. No.: US 2020/0080988 A1
(43) Pub. Date: Mar. 12, 2020

(54) DETECTION DEVICE AND DETECTION METHOD FOR BODY FLUID SEPARATION

(71) Applicant: National Tsing Hua University, Hsinchu (TW)

(72) Inventors: Chao-Min CHENG, Hsinchu (TW); Yi-Tzu LEE, Hsinchu (TW)

(21) Appl. No.: 16/266,779

(22) Filed: Feb. 4, 2019

(30) Foreign Application Priority Data
Sep. 6, 2018 (TW) 107131274

(51) Int. Cl. G01N 33/49; A61B 5/15; B01L 3/00; A61B 5/157

(52) U.S. Cl. CPC G01N 33/491; A61B 5/150755; A61B 5/150251; A61B 5/150351; A61B 5/150343; A61B 5/157; B01L 3/50825

(57) ABSTRACT

The present invention provides a detection device for body fluid separation, which comprises the plurality of bottles, the upper knob member having an upper outer casing and an upper inner casing sleeved within the upper outer casing, and the lower knob member having a lower outer casing and a lower inner casing sleeved within the lower outer casing. When the upper outer casing is tightly coupled to the upper inner casing or the lower outer casing is tightly coupled to the lower inner casing, the bottles do not communicate with each other. When the upper outer casing is separated from the upper inner casing or the lower outer casing is separated from the lower inner casing, the bottles communicate with each other. Accordingly, the detection result can be quickly determined by the detection device and detection method for body fluid separation.

### CROSS REFERENCE TO RELATED APPLICATION

This application claims the benefits of the Taiwan Patent Application Serial Number 107131274, filed on Sep. 6, 2018, the subject matter of which is incorporated herein by reference.

### BACKGROUND OF THE INVENTION

### 1. Field of the Invention

The present invention relates to a detection device and detection method for body fluid separation and, more particularly, to a detection device and a detection method for detecting body fluid after separation.

### 2. Description of Related Art

When people were urgently hospitalized due to intake of harmful substances (such as pesticide poisoning), the common detection method is to first draw blood, test urine and then transfer the blood to the instrument for subsequent testing. However, blood transfer and instrument test have burdensome procedures, and it takes a lot of time to diagnose which pesticide causes poisoning. Thereby, this traditional method may lead to delay in treatment and thus is unsuitable for emergency situations in which the test results need to be obtained immediately. On the other hand, instrumental analysis requires specific technical experts to operate high precision and expensive instruments for inspection. Even though this method is accurate, the disadvantages are long inspection time and higher personnel cost. As the inspection process is too complicated and unable to get data immediately, it is not easy to apply this process in massive inspection.

In view of the drawbacks of existing technology for detecting pesticides in the blood, there is an urgent need to develop various detection devices and methods that can address the demands for rapid detection, simple operation, high sensitivity and low detection cost and simplify the lengthy inspection process of the traditional method, so that poisoned patients admitted to the hospital can be correctly and quickly treated.

### SUMMARY OF THE INVENTION

The object of the present invention is to provide a rapid detection device and implement a detection method through the device, wherein the detection method can quickly judge the detecting result by the naked eye to simplify the lengthy inspection process, and is suitable for on-site instant detection.

To achieve the object, the present invention discloses a detection device for body fluid separation which comprises a first bottle, an upper knob member, a lower knob member, a second bottle and a third bottle. The first bottle has a first opening at one end thereof and a second opening at the other end thereof, wherein the second opening has a first locking structure. The upper knob member comprises an upper outer casing and an upper inner casing sleeved within the upper outer casing. The upper outer casing has a third opening at one end thereof and a fourth opening at the other end thereof facing the first bottle. The fourth opening has a second locking structure connected to the first opening of the first bottle. The lower knob member comprises a lower outer casing and a lower inner casing sleeved within the lower outer casing. The lower outer casing has a fifth opening at the one end thereof facing the first bottle and a sixth opening at the other end thereof facing away from the first bottle. The fifth opening is connected to the first locking structure, and the sixth opening has a third locking structure. The second bottle has a closed end and an opposite open end facing the lower knob member and being formed with a seventh opening. The seventh opening is connected to the third locking structure. The third bottle has a closed end and an opposite open end facing the upper knob member and being formed with an eighth opening. The eighth opening has a fourth locking structure connected to the third opening of the upper outer casing.

The upper outer casing has an upper flange and at least one upper flow channel. The lower outer casing has a lower flange and at least one lower flow channel. The upper inner casing has an upper inner hole, and the lower inner casing has a lower inner hole. When the upper outer casing is tightly coupled to the upper inner casing, the upper flange abuts against the upper inner hole, so that the third bottle does not communicate with the first bottle. When the lower outer casing is tightly coupled to the lower inner casing, the lower flange abuts against the lower inner hole, so that the first bottle does not communicate with the second bottle. When the upper outer casing is separated from the upper inner casing, the upper flange and upper inner hole forms a gap, so that the third bottle communicates with the first bottle. When the lower outer casing is separated from the lower inner casing, the lower flange and lower inner hole forms a gap, so that the first bottle communicates with the second bottle.

In an embodiment of the detection device for body fluid separation, the third bottle is loaded with a detection liquid, and the detection liquid is one or more types of detection liquid.

In a preferred embodiment of the detection device for body fluid separation, the detection liquid is at least one selected from the group consisting of 3,3′,5,5′-tetramethylbenzidine, sodium dithionite, ascorbic acid, and indole-3-acetic acid.

In an embodiment of the detection device for body fluid separation, the first bottle is loaded with a body fluid.

In another embodiment of the detection device for body fluid separation, the body fluid is urine, tissue fluid, or blood.

In another embodiment of the detection device for body fluid separation, the detection device for body fluid separation is a transparent tube.

The present invention provides a detection method for body fluid separation, comprising steps of: providing a detection device for body fluid separation, wherein the detection device for body fluid separation comprises a first bottle, a second bottle, a third bottle, an upper knob member having an upper outer casing and an upper inner casing, and a lower knob member having a lower outer casing and a lower inner casing; placing a body fluid in the first bottle; placing a detection liquid in the third bottle; connecting one end of the upper knob member to the third bottle, and connecting the other end of the upper knob member to the first bottle; connecting one end of the lower knob member to the first bottle, and connecting the other end of the lower knob member to the second bottle; after standing for a period of time, the body fluid being separated into an upper liquid layer and a lower liquid layer; operating the lower knob member to make the lower outer casing and the lower inner casing form a gap, so that the lower liquid layer in the first bottle flows into the second bottle, and the upper liquid layer is retained in the first bottle; operating the lower knob member to make the lower outer casing tightly coupled to the lower inner casing, so that the first bottle does not communicate with the second bottle, thereby separating the upper liquid layer and the lower liquid layer; and operating the upper knob member to make the upper outer casing and the upper inner casing form a gap, so that the detection liquid flows into the first bottle to be mixed with the upper liquid layer, followed by observation of color change of the detection liquid.

In an embodiment of the detecting method for body fluid separation, the body fluid is urine, tissue fluid, or blood.

In another embodiment of the detection method for body fluid separation, the upper liquid layer is plasma.

In another embodiment of the detection method for body fluid separation, the lower liquid layer contains blood cells.

In another embodiment of the detection method for body fluid separation, the detection liquid is at least one selected from the group consisting of 3,3′,5,5′-tetramethylbenzidine, sodium dithionite, ascorbic acid, and indole-3-acetic acid.

In another embodiment of the detection method for body fluid separation, the period of time is 1 to 10 minutes.

In another embodiment of the detection method for body fluid separation, the degree of the color change of the detection liquid is used for half quantitative analysis.

### BRIEF DESCRIPTION OF THE DRAWINGS

FIG. 1 is a perspective view of the detection device for body fluid separation of the present invention;

FIG. 2 is an exploded perspective view of the detection device for body fluid separation of the present invention;

FIG. 3A is a partial schematic diagram of the upper flange being located against the upper inner hole of the present invention;

FIG. 3B is a partial schematic diagram of the lower flange being located against the lower inner hole of the present invention;

FIG. 4A is a partial schematic diagram of the upper flange and upper inner hole forming a gap of the present invention;

FIG. 4B is a partial schematic diagram of the lower flange and lower inner hole forming a gap of the present invention;

FIG. 5 is a flowchart of the detection method for body fluid separation of the present invention;

FIG. 6A is a schematic diagram of the detection device for illustrating the detection method for body fluid separation of the present invention;

FIG. 6B is a schematic diagram of the detection device for illustrating the detection method for body fluid separation of the present invention;

FIG. 6C is a schematic diagram of the detection device for illustrating the detection method for body fluid separation of the present invention;

FIG. 6D is a schematic diagram of the detection device for illustrating the detection method for body fluid separation of the present invention;

FIG. 6E is a schematic diagram of the detection device for illustrating the detection method for body fluid separation of the present invention; and

FIG. 6F is a schematic diagram of the detection device for illustrating the detection method for body fluid separation of the present invention.

### DETAILED DESCRIPTION OF THE PREFERRED EMBODIMENT

The person skilled in the art can easily understand the advantages and effects of the present invention by the contents disclosed in the present specification. However, it should be noted that the following figures are simplified schematic diagrams, the number of components, shape, and size can be changed according to the actual implementation, and the layout state of the component can be more complicated. The present invention may be embodied or applied in various other specific embodiments, and the details of the present invention can be variously modified and changed without departing from the spirit and scope of the invention.

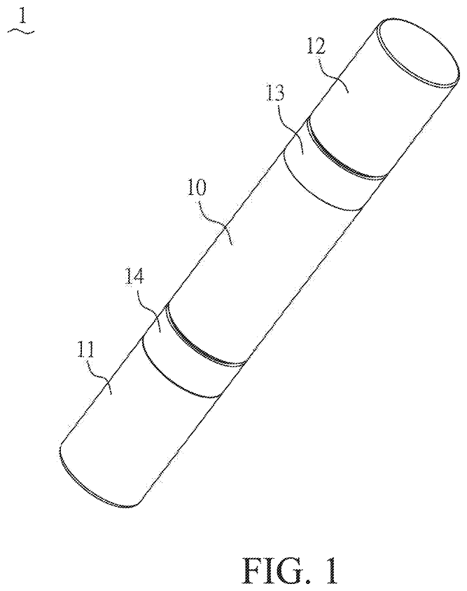

Please refer to FIG. 1, which is an embodiment of a detection device 1 for body fluid separation according to the present invention. As shown in FIG. 1, the detection device 1 for body fluid separation of the present embodiment includes a first bottle 10, an upper knob member 13, a lower knob member 14, a second bottle 11, and a third bottle 12.

In the detection device 1 for body fluid separation of the present embodiment, the upper knob member 13 connects the first bottle 10 and the third bottle 12, and the lower knob member 14 connects the first bottle 10 and the second bottle 11. As a result, the detection device 1 can be used to easily and rapidly detect whether substances to be detected are present in the body fluid. The body fluid can include urine, tissue fluid, or blood. The detection liquid includes 3, 3′, 5, 5′-tetramethylbenzidine (TMB), sodium dithionite, ascorbic acid, indole-3-acetic acid or a combination thereof.

In practical applications, the quantities of the bottles and the knob members are not limited to those shown in the specific embodiment. The person skilled in the art can change the required quantities of the bottles and the knob members according to actual needs. In this specific embodiment, the detection device 1 is illustrated to include three bottles or exemplary description, and blood is taken as an example of the body fluid. Specifically, if the body fluid is blood, three bottles are used. In alternative aspect of the body fluid being urine, the detection device 1 would be configured to include two bottles.

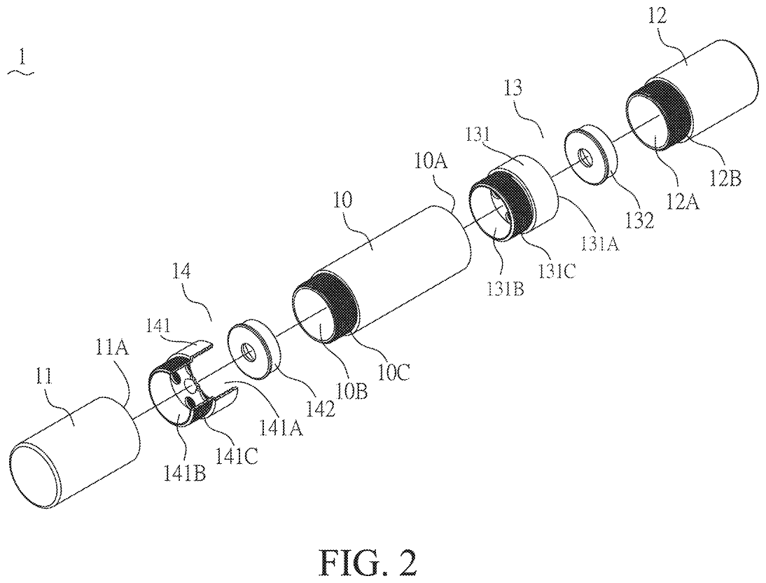

Attention is now directed to FIG. 2 for illustration of the connection between the first bottle 10 and the upper knob member 13. The first bottle 10 has a first opening 10A at one end thereof and a second opening 10B at the other end thereof. The second opening 10B has a first locking structure 10C. In addition, the upper knob member 13 comprises an upper outer casing 131 and an upper inner casing 132 sleeved within the upper outer casing 131. The upper outer casing 131 has openings at its two ends. One end of the upper outer casing 131 is formed with a third opening 131A, and the other end of the upper outer casing 131 faces the first bottle and is formed with a fourth opening 131B. The fourth opening 131B has a second locking structure 131C. The second locking structure 131C of the upper outer casing 131 is connected to the first opening 10A of the first bottle 10 such that the first bottle 10 is connected to the upper knob member 13.

Refer to FIG. 2 again for illustration of the connection between the first bottle 10 and the lower knob member 14. The second opening 10B has a first locking structure 10C. In addition, the lower knob member 14 comprises a lower outer casing 141 and a lower inner casing 142 sleeved within the lower outer casing 141. The lower outer casing 141 has openings at its two ends. One end of the lower outer casing 141 faces the first bottle 10 and has a fifth opening 141A, whereas the other end of the lower outer casing 141 is directed away from the first bottle 10 and has a sixth opening 141B. The sixth opening 141B has a third locking structure 141C. The fifth opening 141A of the lower outer casing 141 is connected to the first locking structure 10C of the first bottle 10 such that the first bottle 10 is connected to the lower knob member 14.

Further, the configurations of the second bottle 11 and the lower knob member 14 are illustrated in more detail below in reference to FIG. 2 again. The second bottle 11 has a closed end and an opposite open end facing the lower knob member 14 and being formed with a seventh opening 11A. Accordingly, the seventh opening 11A is connected to the third locking structure 141C of the lower knob member 14 such that the second bottle 11 is connected to the lower knob member 14.

Still refer to FIG. 2 for illustration the connection between the third bottle 12 and the upper knob member 13. The third bottle 12 has a closed end and an opposite open end facing the upper knob member 13 and being formed with an eighth opening 12A. The eighth opening 12A has a fourth locking structure 12B. Accordingly, the fourth locking structure 12B of the eighth opening 12A is connected to the upper knob member 13 such that the third bottle 12 is connected to the upper knob member 13.

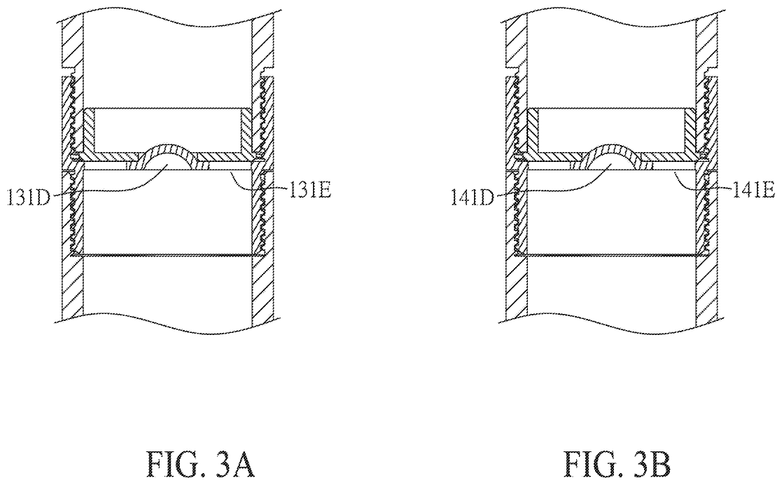

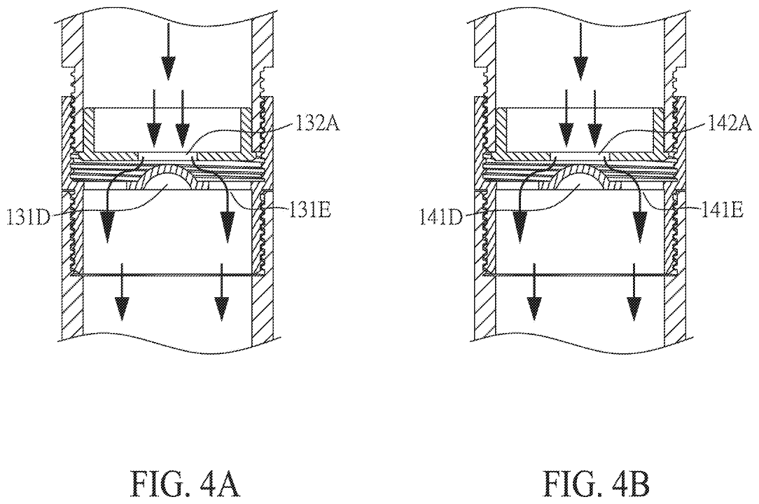

Further, referring to FIG. 3A and FIG. 3B, the upper outer casing 131 has an upper flange 131D and at least one upper flow channel 131E. In actual application, the number and size of the upper flow channel 131E are not particularly limited, and the person skilled in the art can change the number and size of the required upper flow channel 131E according to actual needs. Likewise, the lower outer casing 141 has a lower flange 141D and at least one lower flow channel 141E. In actual application, the number and size of the lower flow channel 141E are not particularly limited, and the person skilled in the art can change the number and size of the required lower flow channel 141E according to actual needs. Next, referring to FIG. 4A and FIG. 4B, the upper inner casing 132 has an upper inner hole 132A, and the lower inner casing 142 has a lower inner hole 142A. The body fluid in the first bottle 10 can be isolated from the detection liquid in the third bottle 12 by closing the upper knob member 13 to make the upper outer casing 131 tightly coupled to the upper inner casing 132. As shown in FIG. 3A, since the upper flange 131D would abut against the upper inner hole 132A, the third bottle 12 and the first bottle 10 do not communicate with each other so as to avoid the body fluid and the detection liquid to flow with each other. Likewise, the body fluid in the first bottle 10 can be isolated and blocked from flowing into second bottle 11 by closing the lower knob member 14 to tightly associate the lower outer casing 141 with the lower inner casing 142. As shown in FIG. 3B, the lower flange 141D would abut against the lower inner hole 142A, so that the body fluid in the first bottle 10 cannot flow into the second bottle 11.

By opening the upper knob member 13, the upper outer casing 131 of the upper knob member 13 is separated from the upper inner casing 132 of the upper knob member 13 so as to permit the detection liquid in the third bottle 12 to flow into the first bottle 10 and to be mixed with the body fluid in the first bottle 10. Specifically, when the upper outer casing 131 is separated from the upper inner casing 132, the upper flange 131D of the upper outer casing 131 and the upper inner hole 132A of the upper inner casing 132 forms a gap (see FIG. 4A). As a result, the third bottle 12 and the first bottle 10 can communicate with each other, and the detection liquid flows to the body fluid. In addition, the body fluid in the first bottle 10 can flow into the second bottle 11 by opening the lower knob member 14. When the lower outer casing 141 is separated from the lower inner casing 142 by opening the lower knob member 14, the lower flange 141D of the lower outer casing 141 and lower inner hole 142 A of the lower inner casing 142 forms a gap (see FIG. 4B) to permit the first bottle 10 to communicate with the second bottle 11. Accordingly, the body fluid in the first bottle 10 flows into the second bottle 11.

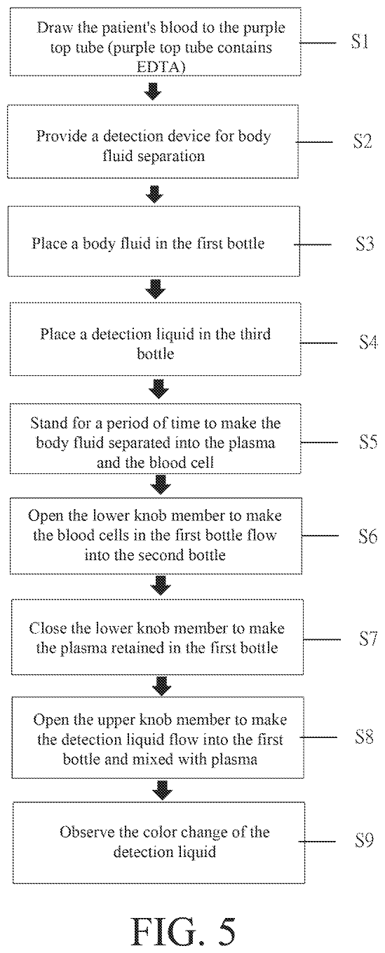

Attention is now directed to steps S1 to S9 of FIG. 5 for description of the detection method for body fluid separation. First, whole blood is injected into a blood collection tube containing EDTA anticoagulant. As EDTA anticoagulant can inhibit blood agglutination, it is employed in this embodiment for whole blood testing. Next, the detection device 1 for body fluid separation of the present invention is provided. The EDTA-containing blood is placed in the first bottle 10, and then the detection liquid is placed in the third bottle 12 (p.s. S3 and S4 can be reversed). After standing for a period of time, the blood would be completely separated into plasma and the blood cells (p. s. if the body fluid is urine, this step can be omitted). Then, the blood cells in the first bottle 10 is directed into the second bottle 11 by opening the lower knob member 14, while the plasma is retained in the first bottle 10 by closing the lower knob member 14. Subsequently, the detection liquid flows into the first bottle 10 by opening the upper knob member 13 to be mixed with the plasma.

Accordingly, the content of the substances to be detected in the plasma can be determined by observation of the color change of the detection liquid and color comparison.

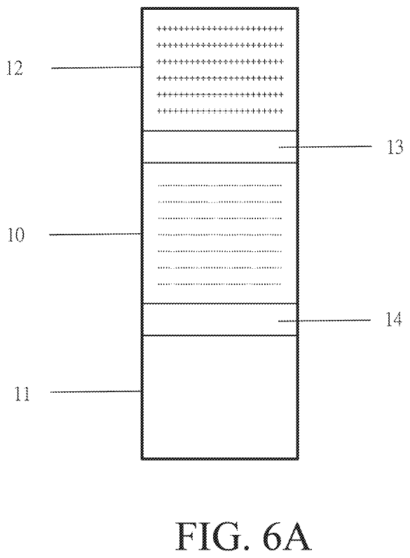

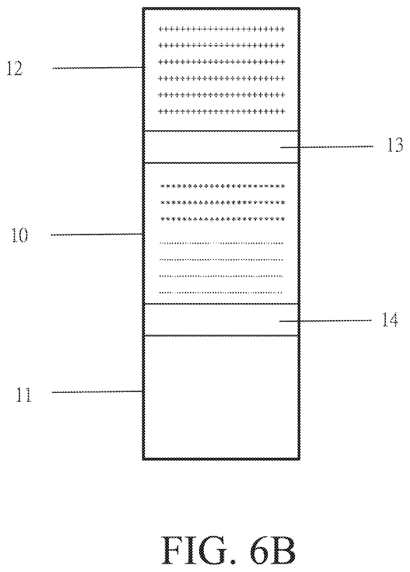

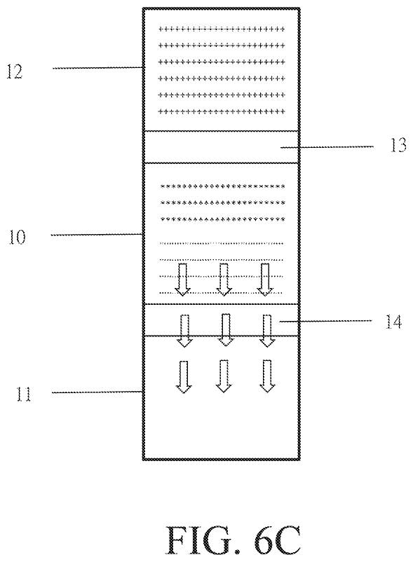

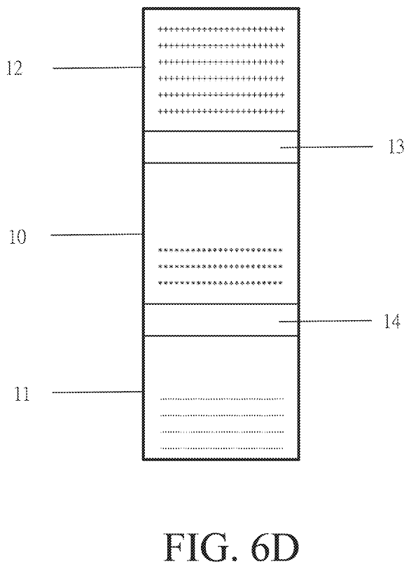

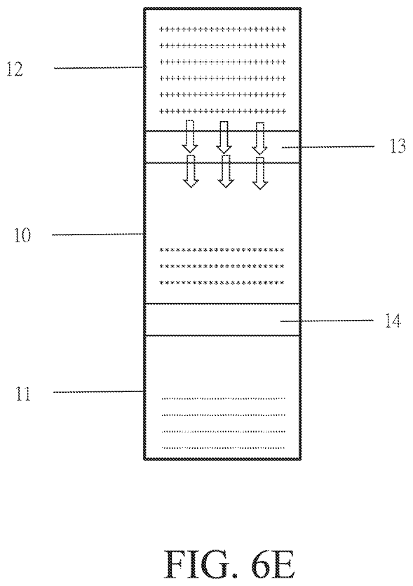

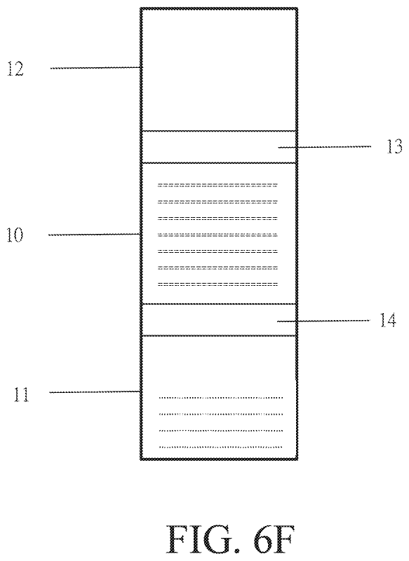

Further, please refer to FIG. 6A to FIG. 6F, which are schematic views for detailed illustration of the detection method. First, the EDTA-containing blood is placed in the first bottle 10, and the detection liquid is placed in the third bottle 12. By the upper knob member 13, the first bottle 10 is assembled with the third bottle 12, as shown in FIG. 6A. After standing for 1 to 10 minutes, the blood is visually divided into two layers, which are upper layer of plasma and lower layer of blood cells, as shown in FIG. 6B. Subsequently, by opening the lower knob member 14 (p.s. the manner of the “opening” step can be changed according to requirements), the lower outer casing 141 can be separated from and the lower inner casing 142 sleeved with the lower outer casing 141, and the lower flange 141D in the lower outer casing 141 and the lower inner hole 142A of the lower inner hole 142 forms a gap to permit the blood cells in the first bottle 10 to flow into the second bottle 11, as shown in FIG. 6C.

Next, referring to FIG. 6D, when all the blood cells flow into the second bottle 11, the lower knob member 14 is closed to make the lower outer casing 141 tightly coupled to the lower inner casing 142. As the lower flange 141D abuts against the lower inner hole 142A, the first bottle 10 cannot communicate with the second bottle 11 so as to separate the plasma from the blood cells.

Subsequently, as shown in FIG. 6E, the plasma and the blood cell are completely isolated in the first bottle 10 and the second bottle 11, respectively. When the upper knob member 13 is opened to separate the upper outer casing 131 from the upper inner casing 132, the upper flange 131D and the upper inner hole 132A would form a gap. Accordingly, the third bottle 12 and the first bottle 10 can communicate with each other to allow flowing of the detection liquid from the third bottle 12 into the first bottle 10.

Finally, as shown in FIG. 6F, after the detection liquid from the third bottle 12 is mixed with the plasma in the first bottle 10, the substance to be detected, if present in the blood, would react with the detection liquid, resulting in color change. Therefore, it is possible to determine whether the blood contains the substance to be detected by the color change after mixing the plasma and the detection liquid.

As illustrated in the aforementioned embodiments, blood is employed in the present invention as an example of the body fluid, and the detection liquid may be 3,3′,5,5′-tetramethylbenzidine (TMB), sodium dithionite, ascorbic acid, indole-3-acetic acid or a combination thereof. The substance to be detected can be organophosphate, carbamate, bipyridyl or a combination thereof. More specifically, the organophosphate comprises methamidophos, malathion, parathionmethyl, parathion, phoxim, dichlorvos, dimethoate, trichlorfon, omethoate and chlorpyrifos; the carbamate comprises carbofuran and methomyl; the bipyridyl comprises paraquat.

Specifically, when the detection liquid is 3,3′,5,5′-tetramethylbenzidine (TMB), the plasma after separation can be detected for the organophosphate; when the detection liquid is sodium dithionite or ascorbic acid, the plasma after separation can be tested for the paraquat; when plasma contains organophosphate or paraquat, the color changes from colorless to blue after adding 3,3′,5,5′-tetramethylbenzidine, sodium dithionite, ascorbic acid or indole-3-acetic acid. According to the embodiment of the present invention, the degree of the color change after mixing detection liquid and the substance to be detected is used as the basis for half quantitative analysis.

In summary, the present invention provides a detection method for body fluid separation using a detecting device for body fluid separation. Each of the components is disposable and inexpensive. It provides a quicker way for the fast-paced emergency rooms to detect the pesticides in the blood. The detection process doesn't take a long time, and the results of detection are determined by the visual inspection (for example, the results may be about whether the blood contains paraquat). Accordingly, the lengthy inspection process can be simplified to make the emergency room run more smoothly and to promote initiation of the subsequent treatment stage correctly and quickly.

The above examples are intended for illustrating the embodiments of the subject invention and the technical features thereof, but not for restricting the scope of protection of the subject invention. Many other possible modifications and variations can be made without departing from the spirit and scope of the invention as hereinafter claimed. The scope of the subject invention is based on the claims as appended.

What is claimed is:

1. A detection device for body fluid separation, comprising:
a first bottle, having a first opening at one end thereof and a second opening at the other end thereof, wherein the second opening has a first locking structure;
an upper knob member and a lower knob member, wherein (i) the upper knob member comprises an upper outer casing and an upper inner casing sleeved within the upper outer casing, (ii) the upper outer casing has a third opening at one end thereof and a fourth opening at the other end thereof facing the first bottle, (iii) the fourth opening has a second locking structure connected to the first opening, (iv) the lower knob member comprises a lower outer casing and a lower inner casing sleeved within the lower outer casing, (v) the lower outer casing has a fifth opening at one end thereof facing the first bottle and a sixth opening at the other end thereof facing away from the first bottle, and (vi) the fifth opening is connected to the first locking structure, and the sixth opening has a third locking structure;
a second bottle, having a closed end and an opposite open end facing the lower knob member and being formed with a seventh opening, wherein the seventh opening is connected to the third locking structure; and
a third bottle, having a closed end and an opposite open end facing the upper knob member and being formed with an eighth opening, wherein the eighth opening has a fourth locking structure connected to the third opening.

2. The detection device for body fluid separation of claim 1, wherein (i) the upper outer casing has an upper flange and at least one upper flow channel, (ii) the lower outer casing has a lower flange and at least one lower flow channel, (iii) the upper inner casing has an upper inner hole, and the lower inner casing has a lower inner hole, (iv) when the upper outer casing is tightly coupled to the upper inner casing, the upper flange abuts against the upper inner hole, so that the third bottle does not communicate with the first bottle, (v) when the lower outer casing is tightly coupled to the lower inner casing, the lower flange abuts against the lower inner hole, so that the first bottle does not communicate with the second bottle, (vi) when the upper outer casing is separated from the upper inner casing, the upper flange and upper inner hole forms a gap, so that the third bottle communicates with the first bottle, and (vii) when the lower outer casing is separated from the lower inner casing, the lower flange and lower inner hole forms a gap, so that the first bottle communicates with the second bottle.

3. The detection device for body fluid separation of claim 1, wherein the third bottle is loaded with a detection liquid.

4. The detection device for body fluid separation of claim 3, wherein the detection liquid is at least one selected from the group consisting of 3,3′,5,5′-tetramethylbenzidine, sodium dithionite, ascorbic acid, and indole-3-acetic acid.

5. The detection device for body fluid separation of claim 1, wherein the first bottle is loaded with a body fluid.

6. The detection device for body fluid separation of claim 5, wherein the body fluid is urine, tissue fluid, or blood.

7. The detection device for body fluid separation of claim 1, wherein the detection device for body fluid separation is a transparent tube.

8. A detection method for body fluid separation, comprising steps of:
providing a detection device for body fluid separation, wherein the detection device for body fluid separation comprises a first bottle, a second bottle, a third bottle, an upper knob member having an upper outer casing and an upper inner casing, and a lower knob member having a lower outer casing and a lower inner casing;
placing a body fluid in the first bottle;
placing a detection liquid in the third bottle;
connecting one end of the upper knob member to the third bottle, and connecting the other end of the upper knob member to the first bottle;
connecting one end of the lower knob member to the first bottle, and connecting the other end of the lower knob member to the second bottle;
after standing for a period of time, the body fluid being separated into an upper liquid layer and a lower liquid layer;
operating the lower knob member to make the lower outer casing and the lower inner casing form a gap, so that the lower liquid layer in the first bottle flows into the second bottle, and the upper liquid layer is retained in the first bottle;
operating the lower knob member to make the lower outer casing tightly coupled to the lower inner casing, so that the first bottle does not communicate with the second bottle, thereby separating the upper liquid layer and the lower liquid layer; and
operating the upper knob member to make the upper outer casing and the upper inner casing form a gap, so that the detection liquid flows into the first bottle to be mixed with the upper liquid layer, followed by observation of color change of the detection liquid.

9. The detection method for body fluid separation of claim 8, wherein the body fluid is urine, tissue fluid, or blood.

10. The detection method for body fluid separation of claim 8, wherein the upper liquid layer is plasma.

11. The detection method for body fluid separation of claim 8, wherein the lower liquid layer contains blood cells.

12. The detection method for body fluid separation of claim 8, wherein the detection liquid is at least one selected from the group consisting of 3,3′,5,5′-tetramethylbenzidine, sodium dithionite, ascorbic acid, and indole-3-acetic acid.

13. The detection method for body fluid separation of claim 8, wherein the period of time is 1 to 10 minutes.

14. The detection method for body fluid separation of claim 8, wherein the degree of the color change of the detection liquid is used for half quantitative analysis.
